(19) United States
(12) Patent Application Publication
(10) Pub. No.: US 2018/0264229 A1
(43) Pub. Date: Sep. 20, 2018

(54) METHOD AND APPARATUS FOR RAPID ASSESSMENT AND TREATMENT OF TRAUMATIC BRAIN INJURY

(71) Applicant: Weinberg Medical Physics, Inc., North Bethesda, MD (US)

(72) Inventor: Irving N. Weinberg, North Bethesda, MD (US)

(21) Appl. No.: 15/923,430

(22) Filed: Mar. 16, 2018

Related U.S. Application Data
(60) Provisional application No. 62/472,482, filed on Mar. 16, 2017.

(51) Int. Cl. A61M 25/01; A61B 5/055; A61B 5/05; A61B 34/00; A61B 5/06

(52) U.S. Cl. CPC A61M 25/0127; A61B 5/055; A61B 5/0515; A61B 2034/2051; A61B 5/062; A61B 2034/731; A61B 34/73

(57) ABSTRACT

Disclosed embodiments enable equipment and methodologies that improve diagnosis and treatment of patients with severe traumatic brain injuries who do not have ready access to surgical theaters

### CROSS-REFERENCE TO RELATED APPLICATION(S)

This application relies for priority on U.S. Provisional Patent Application Ser. No. 62/472,482, entitled “METHOD AND APPARATUS FOR RAPID ASSESSMENT AND TREATMENT OF TRAUMATIC BRAIN INJURY,” filed on Mar. 16, 2017, the entirety of which being incorporated by reference herein.

### FIELD

Disclosed embodiments are directed, generally, to medical therapy, and a brain machine interface that alters and/or diagnoses and or improves biological, cognitive, and/or mood related brain operation.

### SUMMARY

The following presents a simplified summary in order to provide a basic understanding of some aspects of various invention embodiments. The summary is not an extensive overview of the invention. It is neither intended to identify key or critical elements of the invention nor to delineate the scope of the invention. The following summary merely presents some concepts of the invention in a simplified form as a prelude to the more detailed description below.

Some trauma-related injuries, especially to the brain, require treatment application as soon as possible. For example, the mortality of patients with traumatic acute subdural or epidural hemorrhage more than doubles if treatment is delayed more than two hours. However, combat fighters in the field often do not have access to computed tomography or magnetic resonance imaging systems to accurately diagnose such conditions, or to surgical theaters to treat the conditions once diagnosed. As a result brain damage and mortality is greatly increased.

Disclosed embodiments are directed at improving diagnosis and treatment of patients with severe traumatic brain injuries who do not have ready access to surgical theaters.

### BRIEF DESCRIPTION OF FIGURES

A more complete understanding of the disclosed embodiments and the utility thereof may be acquired by referring to the following description in consideration of the accompanying drawings, in which like reference numbers indicate like features, and wherein:

FIG. 1 demonstrates one embodiment of an apparatus including one component 10 at least partially located within a body. The

FIG. 2 demonstrates two possible configurations of the disclosed embodiments as they may be deployed in treatment of diseases of the body (for example, the head) 100.

### DETAILED DESCRIPTION

The description of specific embodiments is not intended to be limiting. To the contrary, those skilled in the art should appreciate that there are numerous variations and equivalents that may be employed without departing from the scope of the present invention. Those equivalents and variations are intended to be encompassed by the present invention.

In the following description of various embodiments, reference is made to the accompanying drawings, which form a part hereof, and in which is shown, by way of illustration, various embodiments in which the invention may be practiced. It is to be understood that other embodiments may be utilized and structural and functional modifications may be made without departing from the scope and spirit of the present invention.

Moreover, it should be understood that various connections are set forth between elements in the following description; however, these connections in general, and, unless otherwise specified, may be either direct or indirect, either permanent or transitory, and either dedicated or shared, and that this specification is not intended to be limiting in this respect.

As an overall introduction to the functionality of the disclosed embodiments, it should be understood that an important concept is that one or more functional particles or components (whose function is dependent on a specific electromagnetic frequency when the particles are in a magnetic field similar to the Earth's field) may be placed in a specific location in a human subject's body. When a device emitting that specific electromagnetic frequency is near the human subject, the particles or components have a beneficial effect on the human subject. It is understood that “functional particle” means a particle that has a particular action, for example delivers a drug or applies a voltage or current or moves.

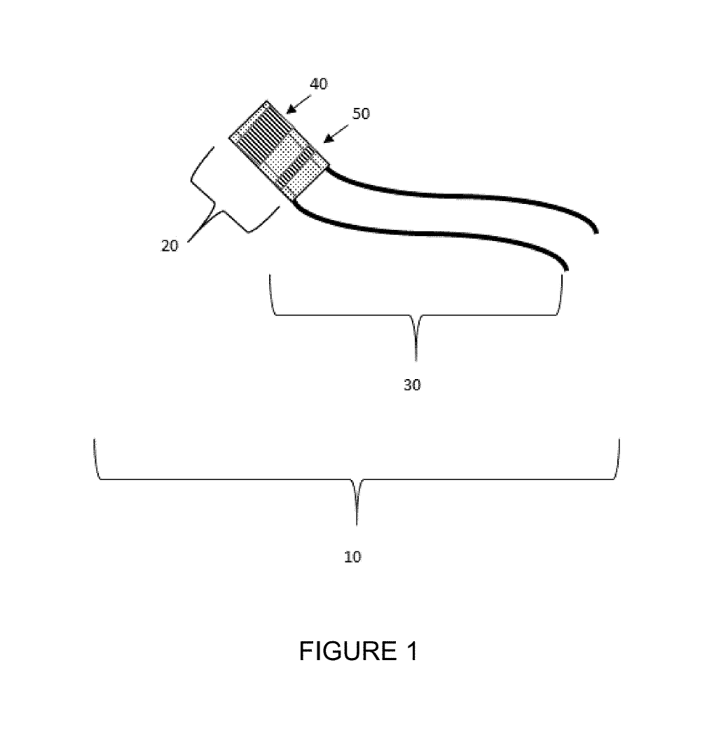

FIG. 1 demonstrates one embodiment of an apparatus including one component 10 at least partially located within a body. The at least one component 10 may include at least one a magnetizable segment 20 and a segment 30 for transporting substances from within the body, shown by example as a catheter.

In accordance with at least one embodiment, at least one portion of the catheter 30 is flexible. The flexible portion of the catheter may be made of, for example, vinyl, red rubber latex, silicon, etc.

In accordance with at least one embodiment, the magnetizable segment 10 may contain at least one sub-segment 40.

In accordance with at least one embodiment, the magnetizable segment 10 may contain at least one other sub-segment 50 having a magnetic easy axis, i.e., the energetically favorable direction of spontaneous magnetization, that is not parallel to sub-segment 40_{[BT1]}.

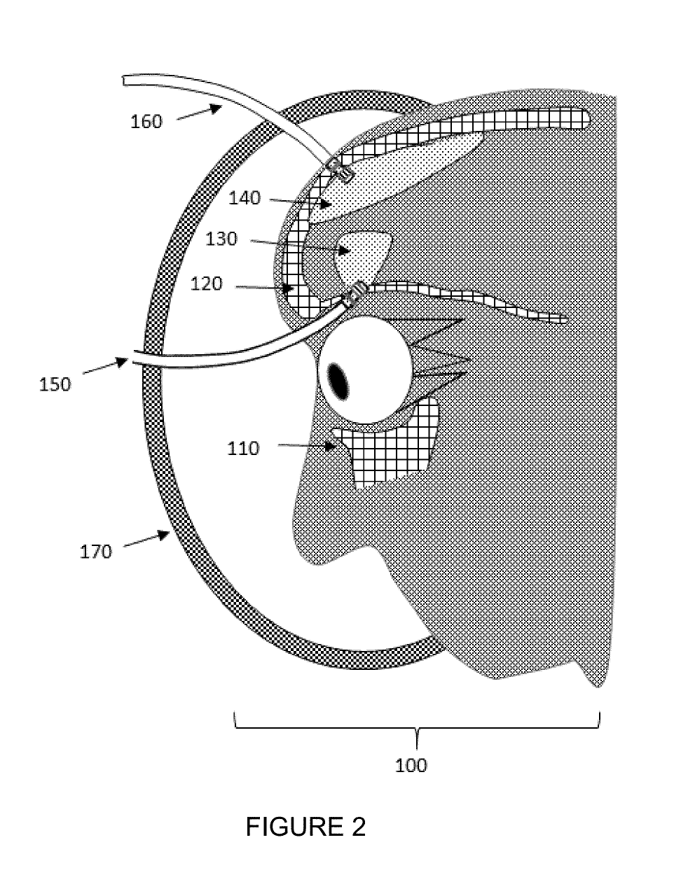

In accordance with at least one embodiment, part of the apparatus (not shown in FIG. 1, but illustrated in FIG. 2, may include one or more coils 170 that are positioned external to the subject's body and is used to manipulate and image component 10.

FIG. 2 demonstrates two possible configurations of the disclosed embodiments as they may be deployed in treatment of diseases of the subject's body (for example, the head) 100. Bony structures 110 (corresponding to the inferior orbital rim) and the cranium 120 are provided to show the orientation of the disclosed components with respect to the head 100. Also shown are representations of an anterior frontal hematoma 130 and a more superior frontal hematoma 140. FIG. 2 shows two configurations of the disposable component 10 (as identified in FIG. 1).

In the configuration shown in FIG. 2, component 10 is shown as structure 150 used to, for example, drain hematoma 130. This may be performed, for example, by inserting structure 150 via the superior orbital fissure above the eye. In another configuration illustrated in FIG. 2, component 10 is shown as structure 160 draining hematoma 140, having been inserted via cranium 120.

Also shown in FIG. 2, is coil 170 representing one or more coils constituting an imaging/propulsion system described below. It is understood that coil 170 may be connected to electrical amplifiers, power supplies and equipment, including display equipment which may transmit images remotely, the images possibly having been collected using the principles of magnetic resonance imaging or magnetic particle imaging. Thus, as shown in FIG. 2, such components 10 may be guided, propelled and/or rotated magnetically via one or more coils schematically illustrated as 170.

Coil 170 may be part of a magnetic field generator, e.g., a magnetic coil and an RF generator or transmitter, wherein the magnetic coil generates a time-varying magnetic field and the RF generator emits radio waves and/or apply a static magnetic field. Accordantly, the coil 170 may be coupled to a power source that may be any type of generator suitable for generating power to be provided to the one or more of the components connected thereto. In an embodiment the emitting device may include a magneto electric material that does not require a coil to emit electromagnetic or magnetic radiation.

Thus, it should be understood that the coil 170 may operate under control of a controller implemented in whole or in part using a computer processor that may be configured assist in performing operations for adjusting levels, timing, locations and types of magnetic fields as described in the incorporated references. Accordingly, software code, instructions and algorithms utilized may be utilized by such a processor and may be stored in a memory that may include any type of known memory device including any mechanism for storing computer executable instructions and data used by a processor. Further, the memory may be implemented with any combination of read only memory modules or random access memory modules, optionally including both volatile and nonvolatile memory.

Alternatively, some or all of the emitting device computer executable instructions may be embodied in hardware or firmware (not illustrated). Further, it should be appreciated that, although not illustrated, the controller may similarly be coupled for communication and control to one or more user interfaces that may include display screens, one or more keyboards, and other types of user interface equipment.

Thus, as illustrated in FIG. 1, disclosed embodiments may provide an apparatus 100 that includes one or more components 10 that may be guided, propelled and rotated magnetically via coils. The one or more components 10 may or may not be disposable, but corresponding to common use of such tools in surgery, such components are referred to herein as being disposable. Such disposable components 10 may include one or more magnetizable regions 40 and 50 provided in a magnetic segment 20, and may also include a section for transporting tissue from within the subject's body to another location.

In accordance with at least one embodiment, this section for transporting tissue from within the subject's body may be a hollow catheter 30 which can evacuate or administer fluids or other substances via the magnetic segment 20 or via segments along the catheter section 30.

The magnetizable segment 20 of the disposable component 10 may be propelled and/or rotated by applying magnetic fields to portions of magnetic segment 20 with component 170, as described in US patent application publication 20170069416 by Lamar Odell Mair, incorporated by reference in its entirety. As disclosed in that reference, one portion 40 could be used to apply a translation force to a device through application of an appropriate magnetic field, while another portion 50 could be used for transmission of a rotational torque to the device through application of a different magnetic field.

The translation force can move component 10 and/or hold component 10 against a subject's body part during drilling through barriers (for example, a cranial bone 120 as shown in FIG. 2). Such a rotation torque may be used to drill a hole in the subject's body in order to gain access to an unwanted lesion in the subject's body, or to disrupt membranes or otherwise liquefy a lesion in the subject's body.

It should be understood that the evacuated portions of a lesion in or on a subject's body may be collected into containers that are not shown in the figures. Examples of such lesions are shown in FIG. 2 as hematomas 130 and/or 140.

As disclosed in patent application publication 20170069416 by Lamar Odell Mair, magnetic fields may be applied to influence sub-sections 40 and 50 of a particle variously to translate the particle and to rotate the particle. Accordingly, one or more magnetic sections of disposable component 10 may be pushed according to the methods described in US patent applications publications 20140309479 and 20160096030 by Aleksandar Nacev (incorporated herein by reference in its entirety).

In accordance with at least some embodiments, high magnetic gradients may be applied by component 170 without causing unwanted nerve stimulation, as described in U.S. Pat. Nos. 9,411,030 and 8,466,680 by Irving Weinberg (incorporated herein by reference in their entirety).

The magnetic gradients may be used for imaging of the human subject's anatomy and particles and/or components and/or propulsion particles and/or components within the human subject's anatomy, as taught in US patent application 20130204120 and U.S. Pat. No. 9,380,959 by Irving Weinberg (incorporated herein by reference in their entirety).

Component 170 may be constructed with electro permanent magnets to reduce space and energy consumption, as taught in US patent application U.S. Non-provisional patent application Ser. No. 15/427,426, entitled “METHOD AND APPARATUS FOR MANIPULATING ELECTROPERMANENT MAGNETS FOR MAGNETIC RESONANCE IMAGING AND IMAGE GUIDED THERAPY,” by Irving Weinberg and Aleksandar Nacev (incorporated by reference in its entirety). With such space and energy reductions, it is understood that the apparatus may be readily transported to (and operated in) remote locations.

In accordance with at least one embodiment, it should be understood that the manipulation of component 10 illustrated in FIG. 1, may be performed remotely by a health or medical aid practitioner with access to images of component 10 and of subject's body part 100 created by component 170 and associated components.

It should also be understood that the component 10 may optionally be fitted with various customized attachments to perform various tasks once the component is positioned within a preferred or target location within the human subject's body, for example suturing tissues, retracting various structures, ligating vessels, etc.

It should be understood that a metallic or otherwise conductive or magnetizable portion of component 10 may optionally be heated in order to cauterize tissues, the heating being implemented by the application or radiofrequency electromagnetic energy or alternating magnetic fields by the one or more coils 170. It should also be understood that component 10 may be moved in order to stimulate nervous tissue under the control of the practitioner.

It should be understood that component 10 may be inserted through natural orifices in the subject's body, for example the nose (for example, to create an intentional cerebrospinal leak), or the supra-orbital fissure 150.

It should be understood that the disposable component 10 may be used to evacuate unwanted lesions by creation of a channel in the subject's body, without the need for a hollow catheter portion.

Although the figures show a human head 100, it is understood that the apparatus and method may apply to other parts of a human subject's body, or to parts of other living or non-living organisms. Although the figures show a single coil 170 as portion of a device for implementing the tasks of manipulating and imaging component 10 and anatomic structures, it is understood that there may be many coils and structures required to accomplish these tasks, and that the use of the one number 170 is a shorthand for the many such coils and structures.

While disclosed embodiments have been described in conjunction with the specific embodiments outlined above, it is evident that many alternatives, modifications and variations will be apparent to those skilled in the art. Accordingly, the various embodiments of the invention, as set forth above, are intended to be illustrative, not limiting. Various changes may be made without departing from the spirit and scope of the invention.

Additionally, it should be understood that the functionality described in connection with various described components of various embodiments may be combined or separated from one another in such a way that the architecture of the resulting system is somewhat different than what is expressly disclosed herein. Moreover, it should be understood that, unless otherwise specified, there is no essential requirement that methodology operations be performed in the illustrated order; therefore, one of ordinary skill in the art would recognize that some operations may be performed in one or more alternative order and/or simultaneously.

Various components of the invention may be provided in alternative combinations operated by, under the control of or on the behalf of various different entities or individuals.

Further, it should be understood that, in accordance with at least one embodiment of the invention, system components may be implemented together or separately and there may be one or more of any or all of the disclosed system components. Further, system components may be either dedicated systems or such functionality may be implemented as virtual systems implemented on general purpose equipment via software implementations.

As a result, it will be apparent for those skilled in the art that the illustrative embodiments described are only examples and that various modifications can be made within the scope of the invention as defined in the appended claims.

1. An apparatus for manipulating and imaging at least one component at least partially located within a subject's body, the apparatus comprising:
one or more magnetic field generators for generating one or more magnetic fields, wherein the one or more magnetic field generators are located external to the subject's body; and
at least one component that contains at least one magnetizable section and at least one section for transporting material from within the subject's body to a position external of the subject's body.

2. The apparatus of claim 1, where the section of the at least one component for transporting material from within the subject's body to the position external of the subject's body is a hollow catheter.

3. The apparatus of claim 1, where the at least one magnetizable section of the component comprises at least two sub-segments with easy axes that are not parallel.

4. A method of accessing unwanted lesions in a subject's body, the method comprising:
imaging at least one component at least partially located within the subject's body using one or more magnetic fields created by a component positioned external to the subject's body; and
manipulating the at least one component at least partially located within the subject's body using one or more magnetic fields created by a component positioned external to the subject's body,
wherein the at least one component at least partially located within the subject's body contains at least one magnetizable section and at least one section for transporting material from within the subject's body.

5. The method of claim 4, where an unwanted lesion is evacuated from the subject's body via the component at least partially located within the subject's body.

6. The method of claim 4, where the component at least partially located within the subject's body is manipulated via remote instructions.

7. The method of claim 4, where the magnetizable section of the component at least partially located within a subject's body drills through barriers within the subject's body.
